(19) United States
(12) Patent Application Publication
(10) Pub. No.: US 2004/0123342 A1
(43) Pub. Date: Jun. 24, 2004

(54) Monocotyledonous plant transformation

(75) Inventors: Adrian Ross Elliott, Auchenflower (AU); Prakash Lakshmanan, Jamboree Heights (AU); Robert Jason Geijskes, Indooroopilly (AU); Nils Berding, Bayview Heights (AU); Christopher Grof, The Gap (AU); Grant Richard Smith, Moggill (AU)

Correspondence Address: MORGAN, LEWIS & BOCKIUS LLP, 1701 MARKET STREET, PHILADELPHIA, PA, 19103-2921, US

(73) Assignees: Sugar Research & Development Corporation; Bureau Of Sugar Experiment Stations; Commonwealth Scientific And Industrial Research Organization

(21) Appl. No.: 10/437,367

(22) Filed: May 12, 2003

Related U.S. Application Data
(63) Continuation of application No. PCT/AU01/01454, filed on Nov. 9, 2001.

(30) Foreign Application Priority Data
Nov. 10, 2000 (AU) PR 1431

(51) Int. Cl. A01H 1/00; C12N 15/82; A01H 5/00

(52) U.S. Cl. 800/278; 800/320.3

(57) ABSTRACT

A method of producing a transgenic monocotyledonous plant includes culturing a thin section explant from a monocotyledonous plant, such as sugarcane, wheat or sorghum, in the presence of an auxin and, optionally, a cytokinin, prior to transformation. Optimally, the thin section is oriented during this pre-transformation culture period of 1-6 days so that a basal surface is substantially not in contact with the culture medium. The cultured explant is then transformed followed by a rest period of 4-15 days in a culture medium without selection agent but comprising an auxin and, optionally, a cytokinin. After this rest period, transgenic plants are selectively propagated from the transformed plant tissue in the presence of a selection agent such as paromomycin sulphate or geneticin. This system provides rapid, efficient generation of transgenic monocotyledonous plants from transformed, non-callus tissue and thereby reduces the likelihood of somaclonal variation among transgenic progeny.

Favorable examination of the present application is respectfully requested at this time.

### FIELD OF THE INVENTION

THIS INVENTION relates to a method of producing transgenic monocotyledonous plants. In particular, his invention applies to producing transgenic plants of the family Gramineae, which includes sugarcane and cereals such as wheat and sorghum, although without being limited thereto.

### BACKGROUND OF THE INVENTION

Many commercially important crops have been the subject of classical brooding aimed at improving agronomically important traits. Crop improvement by such methods is very difficult and usually takes many years, as evidenced by sugarcane, for example, where introduction of novel traits usually takes between 12 and 15 years. Furthermore, crop species which have complex genomes (egg. sugarcane, potato and wheat) often lose useful traits as a result of conventional breeding programs, Thus, genetic engineering has become an attractive and useful alternative to conventional breeding for the introduction of new traits into plants (as reviewed by Briggs & Koziel, 1998, Curr. Op. Biotech. 9 233).

Genetic engineering generally refers to the genetic manipulation of au organism, such as a plant, by way of recombinant DNA technology, so as to modify the genotype of the organism, and thereby create a modified phenotype. Such genetic manipulation typically involves the transformation of an organism with a “transgene” which may confer on the transformed organism a desired trait. Generally, it is desirous that the transgene be stably integrated in the genome of the transformed organism so that the conferred trait is heritable.

A feature of genetic engineering is that the transgene may be obtained from one organism and transferred to another taxonomically disparate organism. This ability to transcend the taxonomic barriers which typically limit the scope of conventional breeding has contributed greatly to the success of genetic engineering.

However, a persistent problem encountered in plant genetic engineering has been somaclonal variation. Somaclonal variation arises from genetic or epigenetic changes caused by unregulated cell proliferation during plant tissue culture. This often results in reduced agronomic performance of transgenic plants compared with the plant(s) from which they are derived. Although in principle, this problem can be overcome by backcrossing, in many situations it is generally desirous to retain the elite characteristics of the variety, without Crier manipulation such as backcrossing.

This problem of somaclonal variation is particularly evident when callus is used as the “target” tissue for gene transfer. In this regard, callus has commonly been used for the purpose of generating transgenic plants. The advantage of callus is that it is has proven to be useful recipient tissue in a wide variety of plants, from which transgenic plants may be readily generated. However, callus produced by unregulated cell proliferation also provides considerable potential for somaclonal variation in transgenic plants generated therefrom.

Thus, in the interests of reducing somaclonal variation, a considerable amount of effort has been aimed at identifying alternative plant tissues suitable for the purposes of plant propagation. Such non-callus based methods are generally referred to as “direct” regeneration methods.

Generally, direct regeneration from non-callus tissue has been readily achieved in dicotyledonous plants, but has met with relatively little success in monocotyledonous plants.

In this regard, some success has been obtained using cytokinins and/or auxins as agents which improve plant regeneration from non-callus tissue Particular examples of the use of auxins and/or cytokinins in the direct regeneration of monocotyledonous plants may be found in Wine & Benda, 1987, Sugarcane 6 14, Irvine et al., 1991, Plant Cell Tissue Organ Cult 26 115, Burner & Grisham, 1995, Crop Sci. 35 875 and Lakshamanan et al., 1996, J. Orch. Soc. Ind 10 31. With regard to these publications, attention is also draw to the efficacy of sectioned explants of non-callus tissue such as leaf sections He & Benda, 1987, supra), or thin sections (TS) of shoot tips, young leaves, inflorcscence and protocom-like bodies (Lakshamanan et al., 1996, supra), for the purposes of direct regeneration.

More particularly, transformation of non-callus monocot tissue has been improved using cytokinins such as N-benzylaminopurine (BAP) or kinetin to assist regeneration from transformed tissue. 1or example, Gambley et al., 1994, Aust. J. Plant Physiol. 21 603 described regeneration of chimeric plants from transformed sugarcane meristem tissue, while in Kamo et a., 1995, Plant Sci. 110 105, stable transformation of Gladiolus was reported where plants wore regenerated from cormel slices cultured in BAP prior to microprojectile bombardment

Reference is also made to International Publication WO 99/15003 which describes transformation of barley meristems cultured in the presence of a cytokinin, copper and, optionally, an auxin.

A recent report by Snyman et al., Abstract 1i: Proceedings of the 4th- International Symposium on In Vitro Culture and Horticultural Breeding (Finland, 2000), claims that transgenic sugarcane can be regenerated from transverse explants derived from immature leaf roll and cultured for at least 2 weeks in medium containing the auxin 2,4-D prior to microprojectile bombardment.

### OBJECT OF THE INVENTION

It has become apparent to the present inventors that despite progress having being made with regard to identifying suitable monocotyledonous plant tissues for transformation, callus is still the most frequently used target tissue for transformation, notwithstanding the problem of somaclonal variation, The present inventors have sought to improve monocot transformation by using a novel combination of plant tissue culture techniques which enhance direct regeneration from transformed plant tissue.

It is therefore an object of the invention to provide a method of producing transgenic monocotyledonous plants.

### SUMMARY OF THE INVENTION

In one aspect, the invention resides in a method of producing a transgenic monocotyledonous plant including the steps of:

(i) culturing a thin section explant from a monocotyledonous plant in the presence of an auxin and, optionally, a cytokinin;

(ii) transforming said explant with an exogenous nucleic acid and, optionally, with a selection marker nucleic acid; and

(iii) selectively propagating a mature transgenic plant from the transformed explant obtained in step (ii).

In one embodiment, the explant is cultured in step (i) so that a basal surface of said explant is not in contact with the culture medium.

Preferably, the duration of culture at step (i) prior to transformation is 1-6 days.

Preferably, at step (ii), the transformed explant is cultured prior to selective propagation at step (iii) for a period of 4-15 days in the absence of a selection agent.

Preferably, at step (iii) the transformed explant is cultured in the presence of a selection agent together with an auxin and cytokinin and then in the absence of an auxin and a cytokinin.

In another aspect, the invention provides a transformed monocotyledonous plant cell or tissue produced at step (ii) of the method according to the first-mentioned aspect. hi yet another aspect, the present invention resides in a transgenic monocotyledonous plant produced according to the method of the first-mentioned aspect. Also contemplated are cells, tissues, leaves, fruit, flowers, seeds and other reproductive material, material useful for vegetative propagation, F1 hybrids, male-sterile plants and all other plants and plant products derivable from said transgenic monocotyledonous plant.

Preferably, the monocotyledonous plant is of the Gramineae family which includes sugarcane and cereals such as wheat, rice, rye, oats barley, sorghum and maize. Other monocotyledonous plants which are contemplated include bananas, lilies, pineapple, tulips, onions, asparagus, ginger, bamboo, oil palm, coconut palm, date palm and ornamental palms such as kentia and rhapis palms.

More preferably, the monocotyledonous plant is sugarcane.

Throughout this specification, it will be understood that “comprise”, “comprises” and “comprising ” are used inclusively rather than exclusively, in that a stated integer or group of integers may include one or more non-stated integers or groups of integers,

### BRIEF DESCRIPTION OF THE FIGURES

FIG. 1: Fluorescence micrographs showing generation of the first GFP-positive transgenic sugarcane shoots from microprojectile-bombarded leaf whorl TS explants. (a) very early stage in the formation of a &-expression apex or embryo-like structure. (b) gfp-expressing embryo or embryo-like structure.

Background fluorescence is due to non-transformed, chlorophyll-containing tissue. (c) an embryo or embryo-like structure with small leaf initials. (d) a larger gfp-expressing shoot showing clearly the developing leaf initials. (e) young gfp-expressing plant (roots not clearly depicted) with two shoots (sugarcane commonly tillers in tissue culture).

FIG. 2: Fluorescence micrographs showing gfp-transformed cells in regenerating sugarcane shoots. (a & b) the appearance of gfp-expressing secondary shoots from apparently chimeric primary transformants. (c, d, e, f & g) chimeric patterns of GFP expression identified during primary regeneration of shoots and the development of transformation methodology for leaf whorl thin sections.

FIG. 3: Regeneration in the presence of the selection agent paromomycin sulphate. Transgenic shoots were regenerated from gfp-transformed leaf whorl TS on 125 mg/L paromomycin sulphate after an initial stage on 100 mg/L. Each of (a)-(h) represents a different transgenic line. The “ligule” regions provide optimal GFP visualization due to minimal background fluorescence contributed by chlorophyll.

FIG. 4: Comparison between transformed (right) and non-transformed (left) plant regenerated from gfp-transformed leaf whorl 1 S.

FIG. 5: Early stage GFP expression in gfp-transformed inflorcscence or leaf whorl thin sections. (a) inflorcscence thin sections showing gfp-expressing cells in central main floral axis (b & c) inflorcscence thin sections showing GFP expressing cells in tissues surrounding central main floral axis. (d-f) inflorcscence thin sections showing gfp-expressing cells ill regenerating areas (usually around the edges of the cut tissues). (g) leaf thin section showing gfp-expressing cells in all areas including outer leaf tissue and in hair cells.

FIG. 6. Development of shoots and plants expressing GFP after transformation of thin sections of inflorcscence or leaf whorl and selection iii 100 mg L⁻¹-paromomycin sulphate. (a) small GFP-positive plant regenerating from leaf whorl. The brightly fluorescent projection to the left is a GFP-positive root.(b) small gfp-expressing shoot/plant regenerating from leaf whorl. (c) regeneration from inflorcscence sections is embryogenic as both roots and shoots fowl during regeneration; A GFP-positive plant is emerging from a single tissue piece removed from an inflorcscence thin section. The tissue from the region surrounding the main floral axis can give rise to more than one plant (d) a small i-expressing shoot viewed from the underside of a petri dish

FIG. 7: GFP-positive transgenic sugarcane shoots/plants regenerated after transformation of thin sections of inflorcscence or leaf whorl. (a-e) show individual gfp-transformed shoot/plant lines. (e) compares transformed shoot (right) with untransformed control shoot (left).

FIG. 8: Regeneration of transgenic microcalli and shoots after bombardment of sugarcane Q165 thin sections (a) Stable transformation demonstrated by the appearance of a small GFP-positive microcalli on a thin section. The thin section was initiated into culture for 3 days, bombarded at 60001da and placed on selection medium after 13 days. () closer view of the tissue in (a). (c) another example of a GFP-positive microcallus. (d), (e) and (f) small GFP-positive shoots forming on thin section pieces at eight weeks after initiation into culture and just over 7 weeks after bombardment.

FIG. 9: Transgenic sugarcane plants. (a) shows potted transgenic sugarcane lines which had been regenerated on antibiotic selection, Each line was GFP-positive. (b) shows mature transgenic sugarcane plants.

FIG. 10: PCR analysis of transgenic sugarcane plant lines. Ethidium bromide stained bands of approximately 750 bp represent amplification of a DNA molecule by primers specific for gfp. The gfp gene was the sgfpS65T construct described in Chiu et al,, 1996, Cur. Biol. 6 325. Untransformed cv. Q165 does not result in band amplification.

FIG. 11: Southern analysis of transgenic sugarcane plant lines. Lane 1: 1Kb DNA Ladder size marker. Lane 2: HindIII-digested transgenic line # 10. Lane 3: HindIII-digested transgenic line #13. Lane 4: HindIII-digested transgenic line # F10. Lane 5: BamHI-digested transgenic line # F10. Lane 6: Uncut transgenic line #P10. Lane 7: HindIII I-digested, untransformed cv. Q165. Lane 8: BamHI-digested, untransformed cv. Q165.

FIG. 12: Transient Ubi-s@S65T expression events recorded after Agrobacterium-mediated transformation experiments of sugarcane cv. Q165 conducted with AGLO. Thin sections were cultured in 4 lM BA, 10 plM NAA and I 0 liM chlorophenoxyacetic acid (CPA).

FIG. 13: Transient expression of s8S65T in wheat cv. Janz transverse sections. Sections were bombarded at 3 days after introduction into culture on EM medium (4.4 g/L MS salts, 20 g/L sucrose, 0.5 g/L casein hydrolysate, I mL/L MS vitamins, 100 mL/L coconut water and 8g/L agar) containing 10)iM CPA and transient expression recorded after another 3 days. (a) - (f) show sgfpS65T expression events and (g) shows a number of GFP-positive cells proliferating,

FIG. 14: Stable expression of Ubi-sgfpS65T-nos in wheat cv. Janz. at 8 weeks after bombardment; (a) and (b) GFP-positive cells in small wheat apices or embryo-like structures which have formed on the surface of the wheat thin sections.

### DETAILED DESCRIPTION OF THE INVENTION

The present invention provides a novel and efficient approach to improve monocotyledonous plant transformation. An important factor is the rapid and direct regeneration of transgenic plants from thin section (TS) explants of tissues such as leaf whorl and inflorcscence, not necessarily through an intermediate callus phase. Preferably, thin sections are cultured so that a basal surface of said explant is substantially not in contact with the culture medium. More particularly, the frequency of explants producing shoots is increased as is the number of shoots produced per explant, when the explant is oriented during culture so that the basal surface is substantially not in contact with the culture medium. This “polarity effect” is also manifested by preferential shoot growth from explants taken distal to the direction of meristematic growth (i.e. non-apical side). The shoots preferably grow from the periphery of the non-apical surface.

Another factor is the period in step (i) where the TS explant is cultured in the presence of an auxin and cytokinin prior to transformation. Similarly, a “rest” period in the presence of an auxin and a cytokinin, without selection agent, after transformation at step (ii) is preferred before selective propagation.

Yet another factor is relatively short duration of culture in the presence of powerful auxins such as 2,4D. Although 2,4-D is not a preferred auxin, it may nevertheless be used according to the present invention. In this regard, the present inventors consider long culture periods in 2,4D (for example 2 weeks or more) to be undesirable by virtue of its potential for inducing callus which in turn may increase the likelihood of somaclonal variation in transgenic plants derived therefrom.

Plant Thin Section Culture

Suitably, the explant is a segment, slice or section of plant tissue.

Preferably, the explant is a thin section (TS) explant.

As used herein, a “TS explant” is a plant tissue segment, slice or section 1.0-10.0 mi in thickness, preferably 1.0-6.0 mm in thickness or more preferably 2.0-3.0 man in thickness.

Suitably, the explant is obtained from plant tissues including leaf spindle or whorl, leaf blade, axillary buds, stems, shoot apex, leaf sheath, internode, petioles, flower stalks, root or inflorescence. A relevant biological property of such suitable tissues is that they contain actively dividing cells having growth and differentiation potential.

In one embodiment the explant is obtained from leaf spindle.

In another embodiment, the explant is obtained from inflorescence.

With regard to iiiflorescence, a preferred source is sugarcane tops in the process of bolting to flower, Typically, sections of inflorescence comprise a main floral axis or stem surrounded by immature rachis branches which will form, or are in the process of forming, floral tissue

As used herein, a “basal surface” of said explant is the surface of said explant distal to the direction of shoot growth of said tissue in an intact plant and proximal to the root system. For example, in the case of sugarcane leaf spindle, the basal surface of the explant is proximal to the apical meristem of the leaf shoot from which the explant is taken. In other words, the basal surface was proximal to the sugarcane stalk in the intact plant.

As used herein “substantially” not in contact with the culture “medium” in te context of the orientation of a basal surface of an explant during culture, means that at least the majority of the basal surface (as hereinbefore defined) does not directly contact the culture medium. This definition includes situations where the explant is cultured with an apical surface in direct contact with the culture medium, in which case the basal surface is oriented distally to the culture medium This definition also includes cases where the explant is placed lengthways horizontally on the medium and neither the basal nor apical surfaces directly contact the medium, except perhaps a portion of the perimeter of each surface which may directly contact the medium.

At step (i) the explant may be cultured for up to 1-6 days prior to transformation. Although this 1-6 day period may be varied or even eliminated depending on the type of plant tissue used, keeping this period relatively short is important for the expression of exogenous nucleic acid and selection marker gene. Hence, this exerts a practical limitation upon the duration of culture at step (i).

The culture medium may include Murashige & Skoog (MS) nutrient formulation (Murashige & Skoog, 1962, Physiologia Plantarum 15 473) or Gamborg's medium (Gamborg et al., 1968, Exp. Cell Res 50 15 1). Preferably, the medium comprises MS formulation. It will be appreciated that the abovementioned media are commercially available, as are other potentially useful media

The medium may further comprise sucrose, preferably at a concentration of 30 g/L. The medium may additionally include agar, preferably at a concentration of 7.5 g/ Thus, it will be appreciated that the TS explant may be cultured in solid or liquid medium.

Additional components of the medium are selected from the group consisting of citric acid (CA) and ascorbic acid Aim). Preferably, the concentration of CA in the medium is 100-200 mg/L, or more preferably I 50 mg/L. Preferably the concentration of AA in the medium is 50-200 Tng/L, or more preferably 100 mg/L

Preferably, the cytokinin is selected from the group consisting of kinetin (KIN), zeatin and N⁶-benzyladenine (BA).

More preferably, the cytokinin is BA or zeatin.

It will be appreciated by the skilled person that there are a variety of other cytokinin, or cytokinin-like compounds which may be useful according to the present invention, for example zeatin, α-isopentyladenosine and diphenylurea

Preferably, the auxin is a-napthaleneacetic acid (NAA) or p-chlorophenoxyacetic acid (CPA).

It will be appreciated by the skilled person that there are a variety of other auxins or auxin-like compounds which may be useful according to the present invention, for example indole-3-butyric acid (IBA), 2,4 dichlorophenoxyacetic acid (2,4D), indole-3-acetic acid (IAA), 2,4,5- trichlorophenoxyacetic acid, phenylacetic acid, picloram, β-napthoxyacetic acid, dicamba and trans-cinnamic acid.

In light of the foregoing, it will be readily apparent to the skilled person tat the most efficacious concentrations of auxin and/or cytokinin applicable to each species of monocotyledonous plant can be determined empirically by cross-testing various concentrations of auxin and cytokinin. In deed, as will be shown hereinafter, although the presence of a cytokinin and/or an auxin is essential to regeneration potential, the optimal concentration of either or both can be tailored according to the plant, or the particular plant cultivar,from which the cultured explant was taken.

If present, preferably the cytokinin is at a concentration in the range 1-20 μM.

More preferably, the cytokinin is present at a concentration in the range 4-12 μM.

Even more preferably, the cytokinin is present at a concentration of 4 μM.

Preferably, the auxin is at a concentration in the range 1-100 dM.

More preferably, the auxin is present at a concentration in the range 10-40 μM.

Even more preferably, the auxin is present at a concentration in the range 10-20 AM.

Expression Constructs

With regard to transformation step (ii), the exogenous nucleic acid is preferably included in an “expression construct”. However, direct isolation of the exogenous nucleic acid and use in microprojectile bombardment is also contemplated, such as described in International Publication WO 00/24244, which is incorporated herein by reference.

The expression construct suitably comprises an exogenous nucleic acid sequence ligated into an expression vector, wherein the exogenous nucleic acid sequence is operably linked to one or more regulatory nucleotide sequences (such as a promoter, enhancer, splice donor/acceptor, terminator and polyadenylation sequence) included in the expression vector that will induce expression of the exogenous nucleic acid in said explant and in transgenic plants regenerated therefrom.

It will be appreciated by the skilled person that the method of the invention is suitable for generating transgenic monocotyledonous plants having any of a variety of desirable traits.

The exogenous nucleic acid sequence may be a nucleic acid isolated from ally organism within the plant or animal kingdoms, bacteria or viruses.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state.

The term “nucleic acid” as used herein designates single-or double-stranded mRNA, RNA, cRNA and DNA inclusive of cDNA and genomic DNA.

A “polynucleotide” is a nucleic acid having eighty (80) or more contiguous nucleotides, while an “oligonucleotide” has less than eighty (80) contiguous nucleotides,

A “probe” may be a single or double-stranded oligonucleotide or polynucleotide, suitably labeled for the purpose of detecting complementary sequences in Northern or Southern blotting, for example.

A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase®.

In one embodiment, the exogenous nucleic acid encodes a polypeptide which confers an agronomically important phenotypic trait upon transgenic monocotyledonous plants produced according to the invention. Such traits may also include disease resistance, for example. Alternatively, the exogenous nucleic acid may confer disease or pest resistance by encoding a sense or anti-sense mRNA corresponding to a viral nucleic acid sequence, such as demonstrated by Joyce et al, 1998, Proc. Aust. Soc, SugarCane Technol. 20 204.

Pest resistance can be engineered by transgenic expression of endogenous genes or by transgenic expression of lectins or proteinase inhibitor genes, such as described by Nutt et al., 1999, Proc Aust. Soc. SugarCane Technol. 21 171.

In another embodiment the exogenous nucleic acid is in the form 5 of a reporter gene. Reporter genes are well known min the art and include chmoramphenicol acetyl transferase (cat; Lindsey & Jones, 1987, Plant Mol. Biol. 10 43), green fluorescent protein and various derivatives thereof (E; Haseloff & Amos, 1995, Trends Genet. 11 328; Elliott et al., Plant Cell Rep. 18 707), neomycin phosphotransferase (nptII; Reiss et al, 1984, Gene 30 21 1), p galactosidase (lacZ; Helmer et al., 1984, BioTechnology 2 520), B-glucuronidase (A; Jefferson e! al. 1987, EMBO J. 6 3301) and luciferase (luc; Ow et al., 1986, Science 234 856; Gambley et at., 1994, supra), each of which references is incorporated herein by reference. The skilled person is also referred to Chapter 9.4 of PLANT MOLECULAR BIOLOGY A Laboratory Manual, Ed. M.S. Clark (Springer-Verlag, Heidelberg, 1997) which is incorporated herein by reference, for examples of specific methods and a general overview of the procedures involved.

In one embodiment, the exogenous nucleic acid is a gfp reporter gene. As used herein, g designates a go nucleic acid, and GFP designates the encoded polypeptide.

By : “polypeptide” is also meant “protein”, either term referring to as amino acid polymer.

A “peptide” is a protein having no more than fifty (50) amino acids.

Proteins, polypeptides and peptides may comprise natural and/or non-natural amino acids as are well known in the art.

Usually, when transgenic expression of a polypeptide is required, the correct orientation of the encoding nucleic acid is 5′-3′ relative to the promoter, for example. However, where antisense expression is required, the exogenous nucleic acid is oriented 3′-5′. Both possibilities are contemplated by the expression construct of the present invention, and directional cloning for these purposes is assisted by the presence of a polylinker.

In one embodiment, the expression construct includes a selection marker nucleic acid to allow selective propagation of plant cells and tissues transformed with an expression construct of the invention. Alternatively, the selection marker is included in a separate selection construct. In either case, one or more regulatory elements, as herein described, may be provided to direct expression of the selection marker nucleic acid.

Suitable selection markers include, but are not limited to, neomycin phosphotransferase II which confers kanamycin and geneticin/0418 resistance (nptII; Raynaerts et al., In: Plant Molecular Biology Manual A9:1-16. Gelvin & Schilperoort Eds (Kluwer, Dordrecht, 1988), bialophos/phosphinothiicin resistance (bar; Thompson el al., 1987, EMBO J. 6 1589), streptomycin resistance (aada; Jones et- al., 1987, Mol. Gen. Genet 210 86) paromomycin resistance (Mauro et al,, 1995, Plant Sci. 112 97), p- glucuronidase (gus; Vancanneyt et al., 1990, Mol. Gen. Genet. 220 245) and hygromycin resistance (hmr or hpt; Waldron et al., 1985, Plant Mol. Biol. 5 103; Perl et al, 1996, Nature Biotechnol 14 624), green fluorescent protein (p; Haseloff & Amos, 1995, sup7a) all of which references are incorporated herein.

Selection markers such as described above may facilitate selection of transformants by addition of an appropriate negative or positive selection agent post-transformation, or by allowing detection of plant tissue which expresses the selection marker by an appropriate assay. hi that regard, a reporter gene such as gfp, nptII, luc or gusA may function as a selection marker.

Preferably, a negative selection agent is used dung selective propagation at step (iii).

Preferably, the negative selection agent is paromomycin sulphate or geneticin.

However, positive selection is also contemplated such as by the phosphomannose isomerase (PS) system described by Wang et al., 2000, Plant Cell Rep. 19 654 and Wright et al., 2001, Plant Cell Rep. 20 429 or by the system described by Endo et al, 2001, Plant Cell Rep. 20 60, for example.

The expression vector of the present invention may also comprise other gene regulatory elements, such as a 3′ non-translated sequence. A 3′ non-translated sequence refers to that portion of a gene that contains a polyadenylation signal and any other regulatory signals capable of effecting, MRNA processing or gene expression. Examples of suitable 3′ non-translated sequences are the 3′ transcribed non-translated regions containing a polyadenylation signal from the nopaline synthase (nos) gene of Agrobacterium tumefaciens (Bevan el al. 1983, Nucl. Acid Res., 11 369) and the terminator for the T7 transcript from the octopine synthase (ocs) gene of Agrobacterium tumefaciens.

Preferably, a nopaline synthase (nos) terminator is utilized..

Examples of transcriptional enhancer elements include, but are not restricted to, elements from the CaMV 35S promoter and octopine synthase (ocs) genes as for example described in U.S. Pat. No. 5,290,924, which is incorporated herein by reference.

Typically, the expression vector of the invention is a plasmid and includes additional elements commonly present in plasmids for easy selection, amplification on, and transformation of the transcribable nucleic acid in prokaryotic and eukaryotic cells, e.g. pUC-derived vectors, pBluescript-derived vectors, pGEM-derived vectors. Additional elements include those which provide for autonomous replication of the vector in bacterial hosts (examples of bacterial origins of replication are the origins of replication of plasmids pBR322, pUC19 and the ColEl replicon which function in many E. coli. strains), bacterial selection marker genes (amp', tet' and kan', for example), unique multiple cloning sites and sequences that enhance transformation of prokaryotic and eukaryotic cells.

Transcription of exogenous nucleic acids and selection marker nucleic acids is suitably controlled by a promoter. Suitable promoters include the CaMV35S promoter, Emu promoter (Last er al., 1991, Theor. Appl. Genet. 81 581, which is herein incorporated by reference) or the maize ubiquitin promoter Ubi (Christensen & Quail, 1996, Transgenic Research 5 213, which is herein incorporated by reference). However, any other promoter functional in monocotyledonous plants would be useful for this purpose.

Preferably, the Hi promoter is used (Christiansen et a..1992. Plant Mol. Biol. 18 675)..

Following tissue culture at step (i), plant tissue is subjected to transformation with, said expression construct and said selection construct.

In one embodiment, transformation is by microprojectile bombardment, for example as described by Pranks & Birch, 1991, Aust. J. Plant. Physiol., 18:471; Gambley el al., 1994, supra; and Bower et al, 1996. Molecular Breeding, 2:239, which are herein incorporated by reference.

The basis of a preferred method of microprojectile bombardment is provided in Bower et al, 1996, supra.

In another embodiment, transformation is Agrobacterium-mediated. Examples of Agrobacterium-mediated transformation of monocots are provided in U.S. Pat. No. 6,037,522, Hiei et at, 1994, Plant Journal 6 271 and Ishida et al., 1996, Nature Biotechnol. 14 745 in relation to various cereals, Arencibia Et al, 1998, Transgenic Res. 7 213 and Elliot er al, 1998 Aust. J. Plant Physiol. 25 739 in relation to sugarcane and International Publication WO99/36637 in relation to pineapples.

Accordingly, persons skilled in the art will be aware that a variety of other transformation methods are applicable to the method of the invention such as liposome-mediated (Abokas et al. 1987, Heriditas 106 129), laser- mediated (Guo et al., 1995, Physiologia Plantarum 93 19), silicon carbide or tungsten whiskers (U.S. Pat. No. 5,302,523; Kaeppler et al. 1992, Theor. Appl. Genet 84 560), virus-mediated (Brisson et al., 1987, -Nature 310 511), polyethylene-glycol-mediated (Paszkowski et al., 1984, EMBO J. 3 2717) as well as transformation by microinjection (Neuhaus et al., 1987, Theor. Appl. Genet. 75 30) and electroporation of protoplasts (Fromm et al., 1986, Nature 319 791).

It will be appreciated that the aforementioned references are non-limiting examples of suitable methods, all of which references are incorporated herein.

Following transformation, there is preferably a period of 4-15 days or more preferably 7-12 days where the transformed tissue is cultured in the presence of an auxin and a cytokinin, preferably without selection agent prior to selective propagation. The auxin and cytokinin, and their respective concentrations, are as hereinbefore described.

Selective Propagation of Transformants

Selective propagation at step (iii) preferably occurs in two distinct stages.

Preferably, in first stage (a) a selection agents preferably paromomycin sulphate, is present ill the culture medium together with an auxin and cytokinin as hereinbefore described.

The preferred concentration of paromomycin sulphate at stage (a) is 75-150 mg/L.

The preferred duration of step (a) is about 3-4 weeks.

In second stage (b) a selection agent, preferably paromomycin sulphate, is present in the culture medium in the absence of an auxin and cytokinin.

The preferred concentration of paromomycin sulphate at stage (b) is in the range 100 mg/L to 150 mg/L.

The preferred duration of stage (b) is 5-8 weeks, although this period may be extended to promote extensive root formation on media containing 150 mg/paromomycin sulphate.

It will be appreciated that selective propagation may be performed using, any of a. variety of selection agents other than paromomycin sulphate including hygromycin, Geneticin®/G418, 4=kanamycin, bialaphos, streptomycin as already described, Furthermore, it will be appreciated that selective propagation can be performed where the expression construct includes the selection marker nucleic acid and where the selection marker nucleic acid is included in a separate selection construct.

Whichever method is used, the selectively propagated tissue is observed for shoot and/or root growth, )ni cases where an exogenous nucleic acid encoding GFP is used for transformation, GFP expression can be monitored in the transgenic plantlets and shoots, particularly when the plantlets are chimeric.

The transgenic plantlets (preferably at 5-10 cms in length) are then 5 propagated in soil or a soil substitute to promote growth into a mature transgenic plant. Preferably, propagation of transgenic plants from plantlets at step (iii) is performed in Perlite, peatmoss and sand (1:1:1) under glasshousc conditions.

Detection of Transgene Expression

In one embodiment, the transgenic status of plants produced according to the method of the invention may be ascertained by measuring transgenic expression of a polypeptide encoded by the exogenous nucleic acid.

Transgene expression can be detected by using antibodies specific for the encoded polypeptide:

(i) in an ELISA such as described in Chapter 11.2 of CURRENT PROTOCOLS IN MOLECULAR BIOLOGY Eds. Ausubel et al. (John Wiley & Sons Inc. NY, 1995) which is herein incorporated by reference; or

(ii) by Western blotting and/or immunoprecipitation such as described in Chapter 12 of CURRENT PROTOCOLS IN PROTEIN SCIENCE Eds. Coligan et al. (John Wiley & Sons Inc. NY, 1997) which is herein incorporated by reference.

Protein-based techniques such as mentioned above may also be found in Chapter 4.2 of PLANT MOLECULAR BIOLOGY: A Laboratory Manual, sup?a, which is herein incorporated by reference.

In another embodiment, transgenic plants of the invention may be screened for the presence of the exogenous nucleic acid transgene and/or the selection marker nucleic acid according to expression of a corresponding mRNA. In order to detect the exogenous nucleic acid transgene Southern hybridization and/or PCR may be employed. To measure mRNA expression, RT-PCR and/or Northern hybridization may be employed,

PCR is a technique well know in -the art, but for a detailed description and exemplary methods the skilled person is directed to Chapter 15 of CURRENT PROTOCOLS IN MOLECULAR BIOLOGY Eds. Ausubel et al., supra, and Chapter 2 of PLANT MOLECULAR BIOLOGY A Laboratory Manual, supra which are incorporated herein by reference

For examples of RNA isolation and Northern hybridization 5 methods, the skilled person is referred to Chapter 4 of CURRENT PROTOCOLS E MOLECULAR BIOLOGY (Eds. Ausubel et al; John Wiley & Sons Inc., 1995).

Southern hybridization may also be used to verify integration of the exogenous nucleic acid and/or the selection marker into the monocotyledonous plant genome. Southern hybridization techniques are well known to those skilled in the art, and have been described, for example, in CURRENT PROTOCOLS E MOLECULAR BIOLOGY (Eds. Ausubel er al.; John Wiley & Sons Inc., 1995) at sections 2.9A-B and 2.10, which are herein incorporated by reference.

So that the invention may be understood in more detail, the skilled person is referred to the following non-limiting examples.

### EXAMPLE1

### General Media and Culture Conditions

Murashige & Skoog (MS) nutrient formulation supplemented with 30 g/L sucrose and 7.5 g/L Difco agar was used as the basal culture medium. Basal medium was, for certain phases of culture as will be described below, enriched with the cytokinin N⁶-benzylaminopurine (BA) and the auxin a-napthaleneacetic acid NAA) together with anti-oxidants such as citric acid (CA), ascorbic acid (AA), or dithiothreitol (DTT). The pH of the medium was adjusted to 5.7 before autoclaving at 11kPa for 20 minutes at 120° C. TS explants were cultured in various orientations either in tissue culture dishes (90×14 mm) with 40 ml agar- solidified medium or in a 100 mL baby food jar containing 40 ml liquid medium, or on membrane rafts with flotation kept in a polypropylene container with 40 ml liquid medium. Liquid cultures were agitated continuously on a gyratory shaker at 120 rpm. All cultures were incubated at 25-280° C. under 16 hr photoperiod provided by cool, white fluorescent tubes, Subculturing was carried out at least once a week, or more frequently if medium or TS turned brown due to phenolic exudation.

Osmoticum medium was prepared using 0.2 M mannitol and 0.2 M sorbitol.

With regard to plant tissue culture and regeneration, reference is made to PCT/AU01/00483 which provides additional information pertinent to these facets of the invention.

### EXAMPLE 2

### Sugarcane Thin Sections

Sugarcane leaf sheath tissue taken from just above the meristem was taken from cultivar Q165 plants. Transverse thin section explants (2-3 mm) of leaf whorl were obtained from the harvested tissue. These are generally referred to as leaf thin sections.

Sugarcane (cultivar Q165) tops in the process of bolting to flower were harvested for inflorescence tissue. Transverse sections were made to produce 2-3 mm thin sections which contained a heterogeneous mixture of floral and leaf cells. These are generally referred to as inflorescence thin sections

### EXAMPLE 3

### Preliminary Experiments to Determine Culture Conditions for Leaf Whorl

Preliminary experiments using sugarcane leaf thin sections showed that although shoots were produced by TS explants under a range of culture conditions, it was clear that TS explants having their ba surface not contacting the medium (“top down”) produced a significantly greater number of shoots. Also, the only TS explants which produced large numbers of shoots (>20 per explant) after 6 or 8 weeks of culture were those where the explant was oriented so that the basal surface did not contact the medium (“top down”). This trend was also evident, although less marked, after 5 weeks of culture.

Variations in section thickness showed that 1-2 mm, 2-3 mm and 5-6 mm thick sections may be used, although 2-3 mm sections are preferred for the purpose of microprojectile bombardment.

Cross-testing of NAA and BA concentrations was also performed using TS leaf whorl explants obtained from the Q187 and Q124 sugarcane cultivars. In Q187, 4 μM BA was clearly more efficient than 8 μM BA, but with Q124 there was no clear preference for 4 μM BA over8 μM BA by an% of the regeneration criteria examined. However, higher concentrations of NAA (10-40 μM) promoted a slightly hither percentage of explants producing shoots. There was no clear trend in terms of the number of shoots produced with regard to either NAA or BA concentration, Generally, 4 μM BA was preferred at any given concentration of NAA, while NAA was optimal when present in culture medium at a concentration of 10 μM.

A detailed summary of optimal cytokinin and auxin concentrations and thin section polarity is provided in PCT/AU01/00483.

### EXAMPLE 4

### Pre-Transformation Tissue Culture

Based on the above preliminary studies, it was found that placement of TS explants so that a basal surface thereof did not contact the culture medium (e.g. with the apical surface in contact with the medium) was optimal during culture. The leaf whorl and inflorescence TS explants were therefore cultured in this fashion on solid MS medium/agar in the presence of 4 μM BA and 10 μM NAA for a period of 2-4 days, during which time there was no detectable shoot development Approximately 3-4 hr prior to microprojectile bombardment, TS explants were placed onto solid MS medium comprising 4 μM BA,10 μM NAA and osmoticum (see Example 6).

### EXAMPLE 5

### Expression Constructs

The exogenous nucleic acid used in all transformation experiments was a reporter gene encoding GFP inserted into pGEM.Ubi (Elliott et al., 1999, supra). For selection, pUbi.KN was used throughout as a separate selection construct. pUbi.KN includes the nptII selection marker gene driven by the maize ubiquitin promoter (Ubi) and the nos 3′ region. The expression construct and selection construct were used as a 1:1 (v/v) DNA mixture for transformation.

### EXAMPLE 6

### Microprojectile Bombardment

The method was essentially as described previously in Bower et al., 1996, supra. Helium pressure (3000 kPa) was used to deliver microprojectiles with a 0.1 msec 5 solenoid opening duration, and 4,ul of DNA-coated microprojectiles used per shot, as follows:

1. Thin sections were placed in a circle (˜3 cm diameter). Some experiments incorporated osmoticum treatments (0.2 M mannitol and 0.2 M sorbitol) for 8 hours total on the day of bombardment.

2. Tungsten microparticles (1-121im dia) were sterilized in a volume of EtOH (10 μl/mg tungsten), vortexed, centrifuged for ˜10 secs and the 1EtOH replaced with an equal volume of sterile dH₂O, The washing step was repeated twice prior to resuspension in the same volume of water.

3. The DNA/microprojectile mix was prepared as follows. Vortexing was performed briefly between additions.

| Precipitation mix | vol. |
|---|---|
| 100 μg/μl tungsten | 50 μl |
| DNA (˜1 μg/μl) | 10 μl |
| 2.5 M CaCl₂ | 50 μl |
| 100 mM spermidine | 20 μl |
| | 130 μl |

4. The mixture was allowed to settle on ice for 5 minutes, 100 μl removed and the remainder resuspended.

5. Thin sections were bombarded with 4 μl DNA-microprojectile mix after 3-4 hr and then left on osmoticum treatment for 3-4 hr post-bombardment.

### EXAMPLE 7

### Post-Transformation Tissue Culture

A number of preliminary experiments were performed to detennije parametens which provided optimal regeneration from transformed tissue while providing optimal selection of transformants.

Transfer of leaf TS explants to medium contaiiaig 100 mg/L paromomycin sulphate at 3 days after bombardment resulted in no shoot regeneration. Similar results were obtained when bombarded TS explants were transferred to medium containing even low levels of selection agent. Experiments to determine the effect of selection on regeneration revealed that following bombardment, a “rest” period of 5-10 days in the absence of selection agent, but in the presence of 4 μM BA and 10 μM NAA, was optimal.

During this rest period, usually 2 days after bombardment, the explants were assessed by fluorescence microscopy to detect transient GFP expression. Following this rest period, a lower. less toxic level of selection agent (for example 75 mg/L paromomycin sulphate for 24 weeks) was included in the medium followed by higher levels (100 or 125 mg/L paromomycin sulphate for approximately 4 weeks). It was also found that this latter stage of selective propagation was best performed in the absence of an auxin and cytokinin.

### EXAMPLE 8

### Detection of Transgenic Plants and Transformed Tissue

GFP-positive transgenic shoots regenerated in the presence of 125 mg/L paromomycin sulphate produced GFP-expressing roots in this level of selection agent. This was routinely checked prior to transferring plantlets to a glasshouse.

GFP fluorescence microscopy was performed essentially as described i. Elliott et al., 1998, supra, which is incorporated herein by reference.

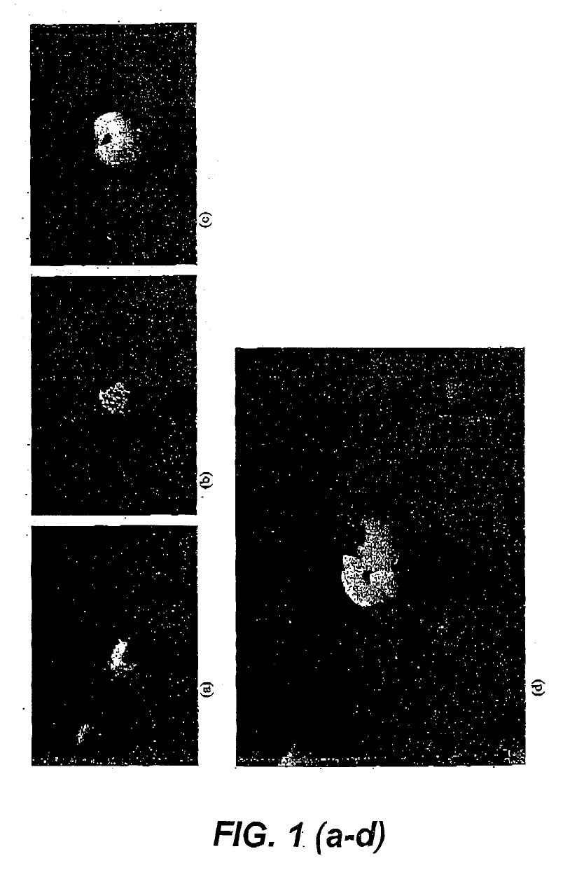

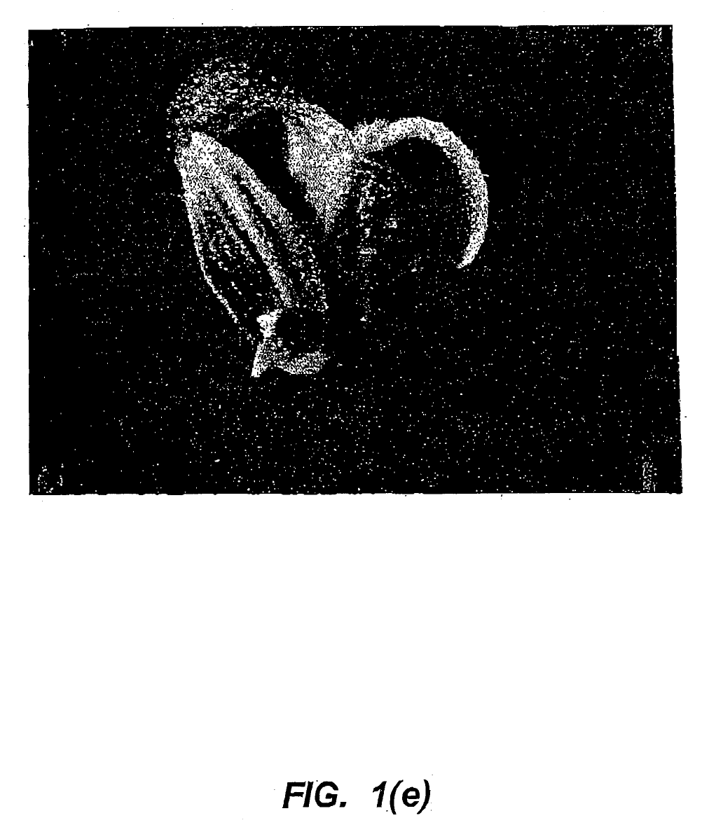

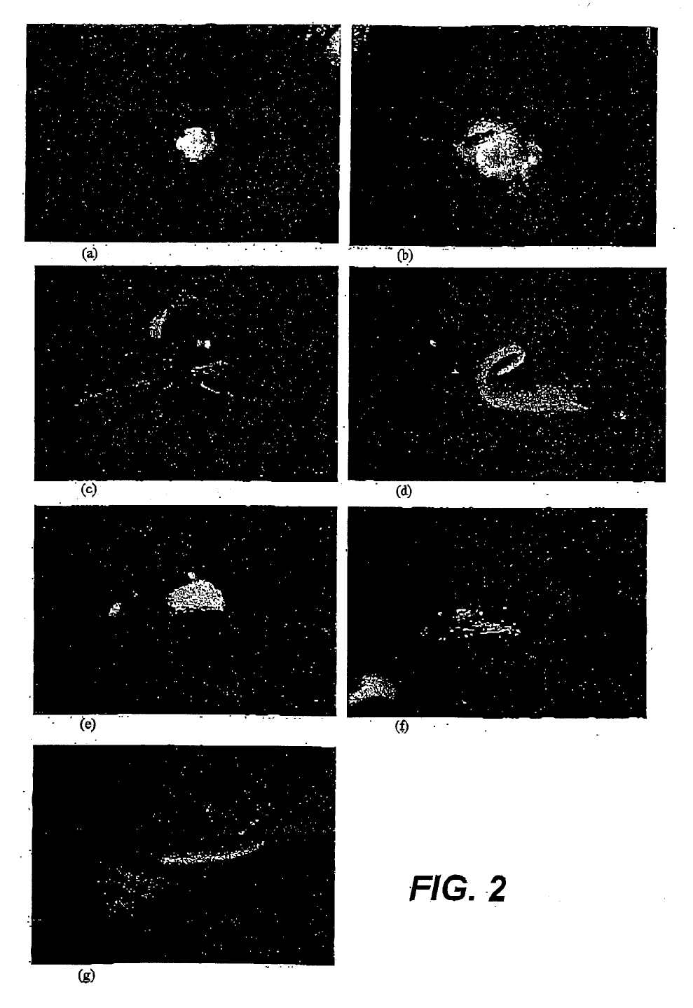

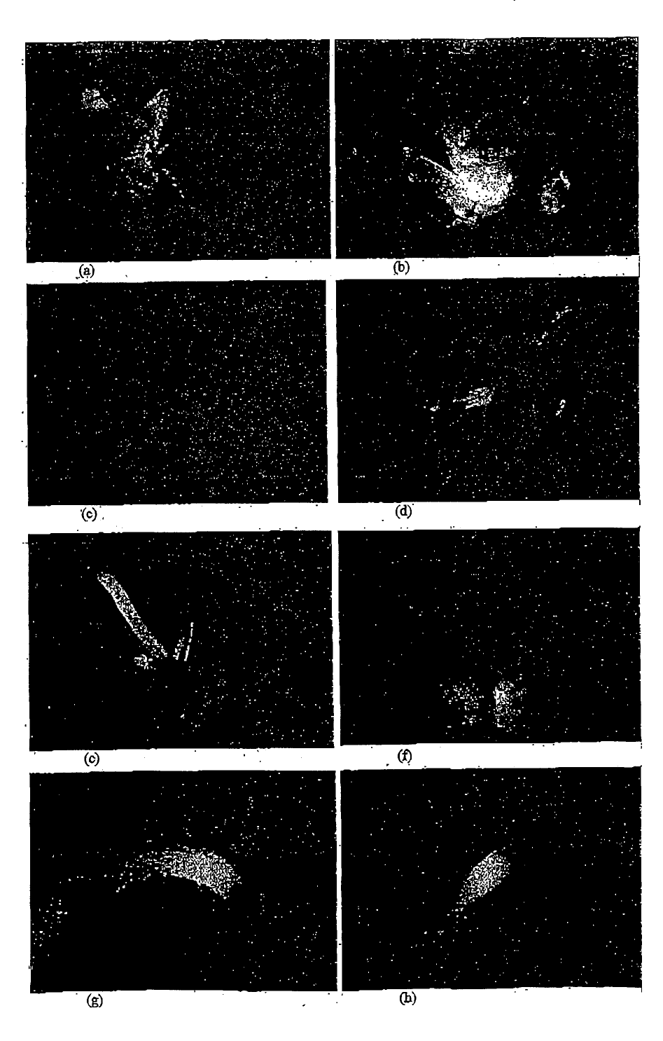

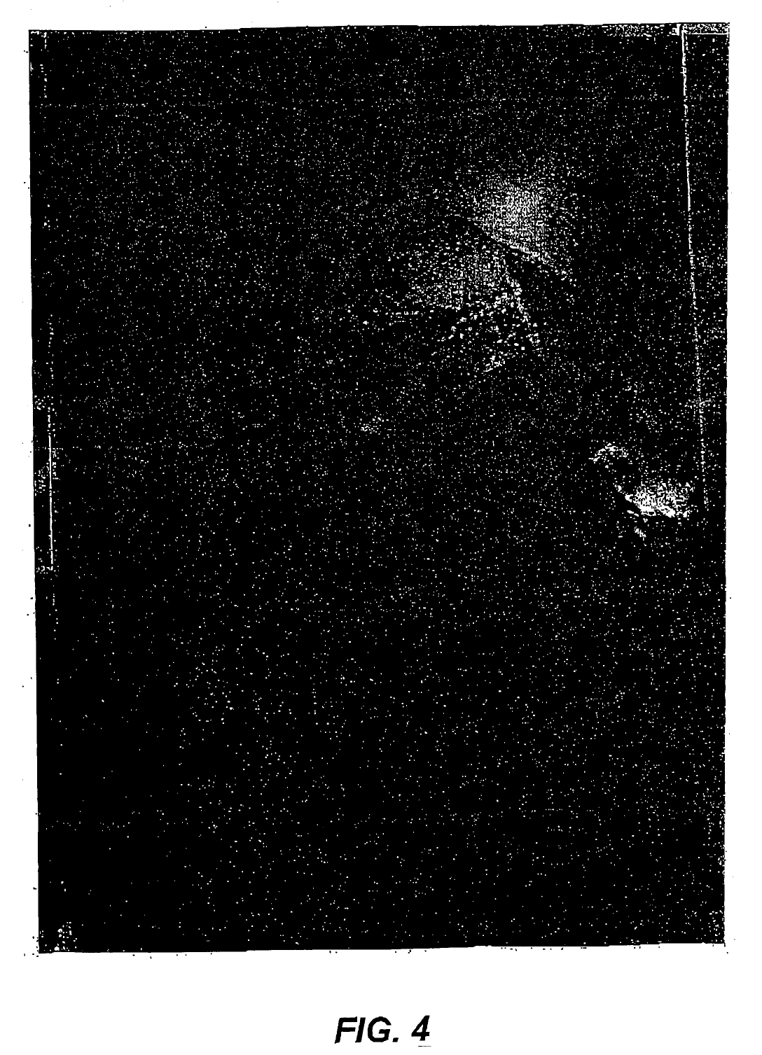

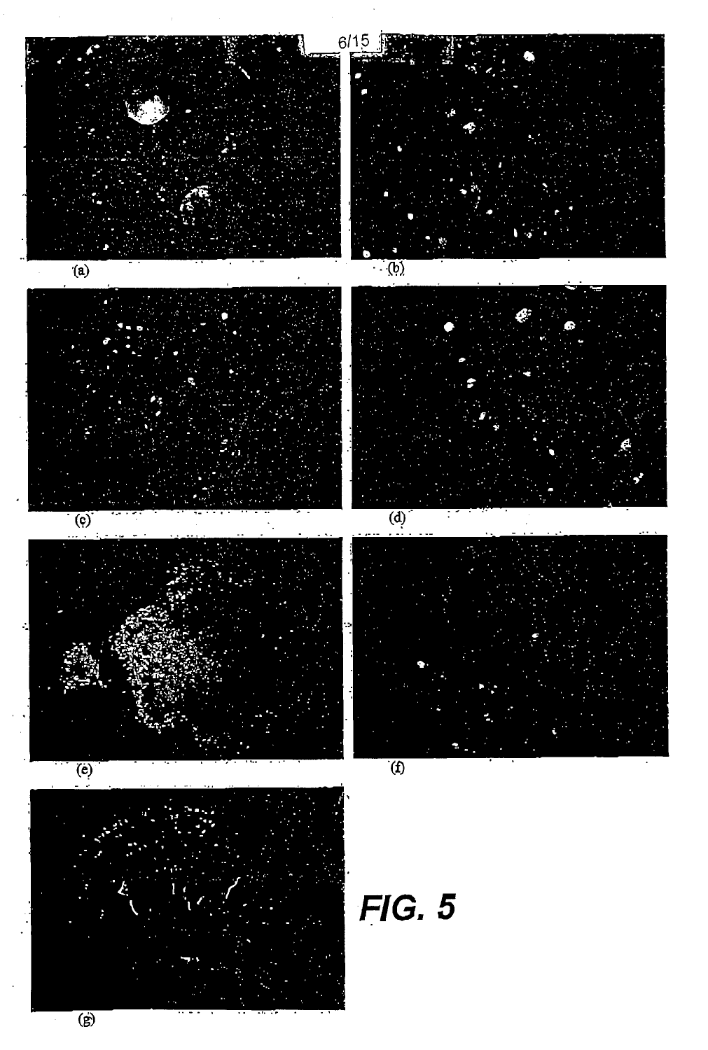

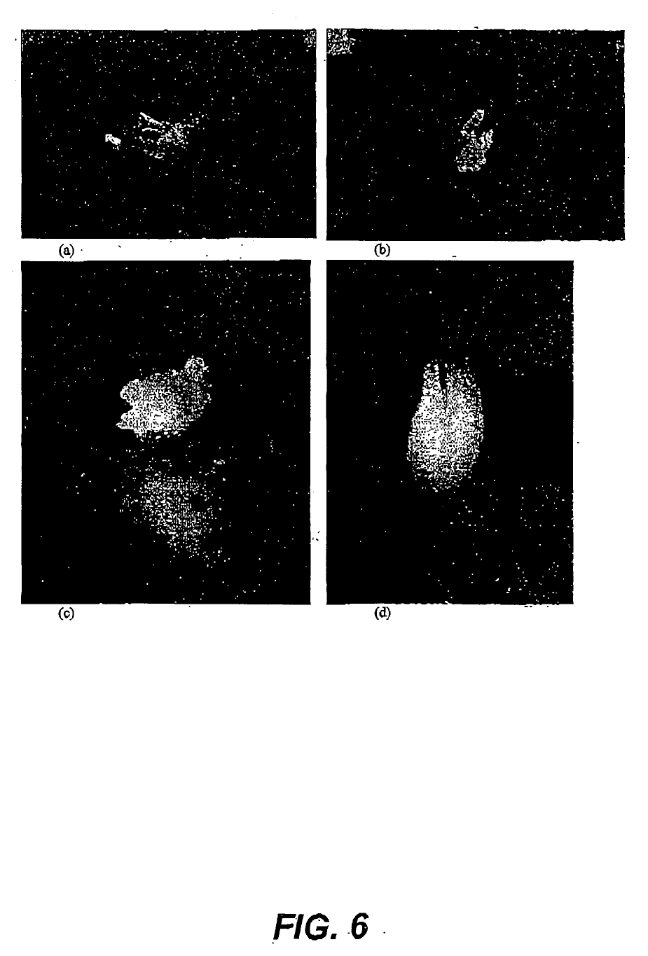

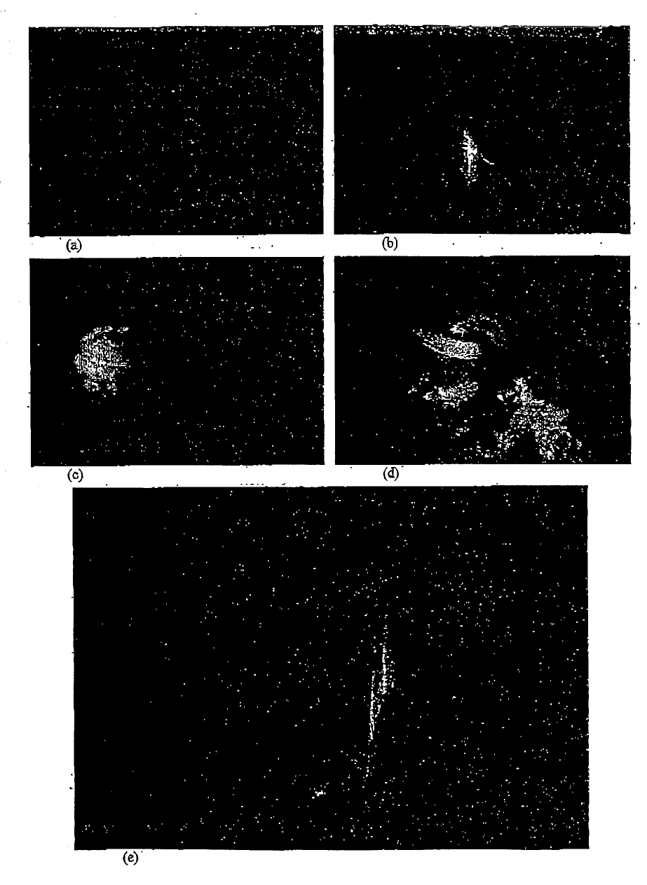

In FIGS. 1-4 there are provided a number of examples of gfp-transformed plant and shoot lines derived from transformed thin sections of leaf whorl. In FIGS. 5-7, examples of gfp-transformed plant and shoot lines derived from gfp-transformed thin sections of leaf whorl and inflorescence are shown.

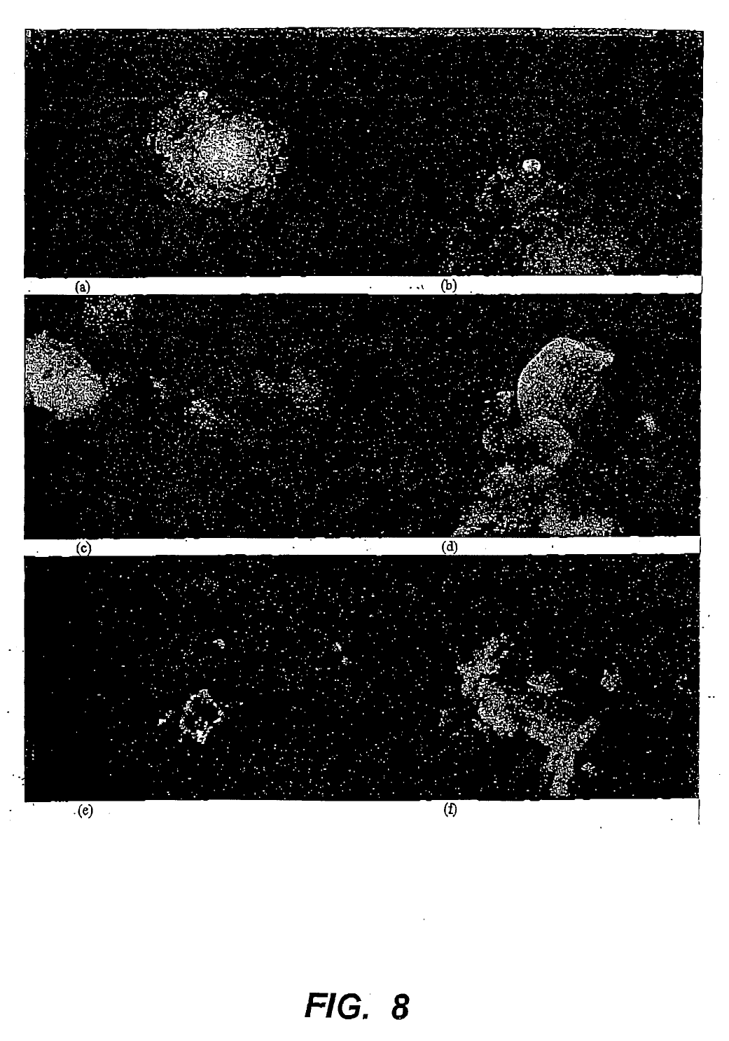

Clearly, both leaf and inflorescence sections were transformable and capable of expressing GFP. in shoots, roots and plants regenerated therefrom. However, a distinction between leaf whorl and inflorescence thin sections was that in some cases regeneration from bombarded inflorescence was embryogenic. That is, GFP-positive shoots with roots were regenerated directly from inflorescence sections, rather than by organogenic regeneration as was typical for leaf whorl. Generally, when regenerating from inflorescence sections, GFP-positive shoots and plants were seen arising from tissues surrounding the main floral axis. li FIG. 8, another type of regeneration is shown involving microcallus formation.

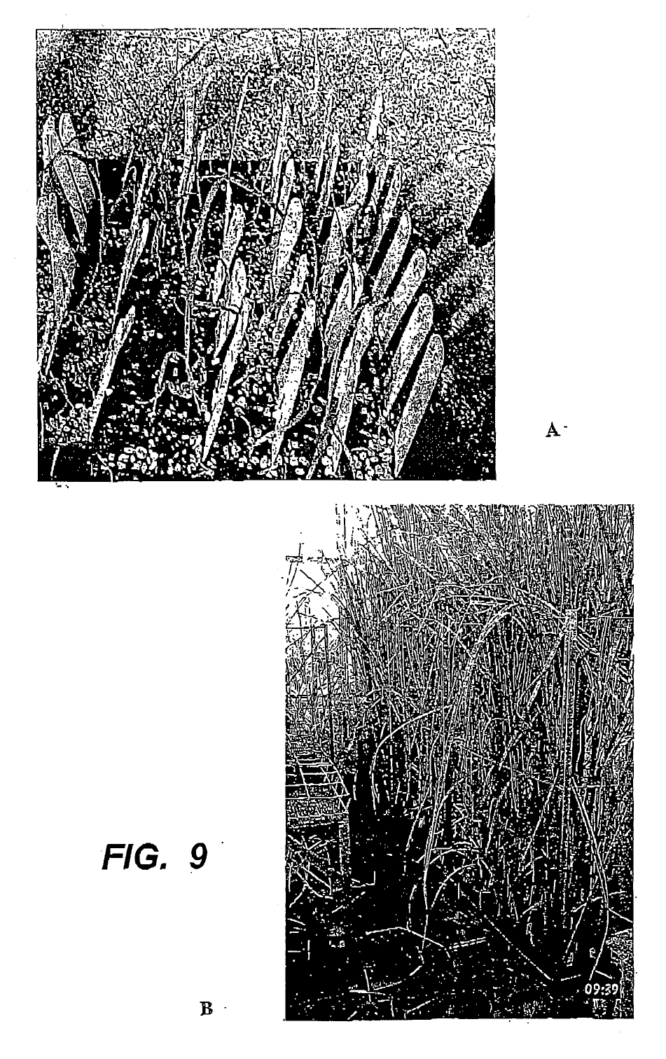

To data, a number of mature, gfp-expressing transgenic sugarcane lines have been produced as shown in FIG. 9.

### EXAMPLE 9

### Confirmation of the Presence of Introduced sgfpS65T Gene in Transgenic Plants by PCR Detection

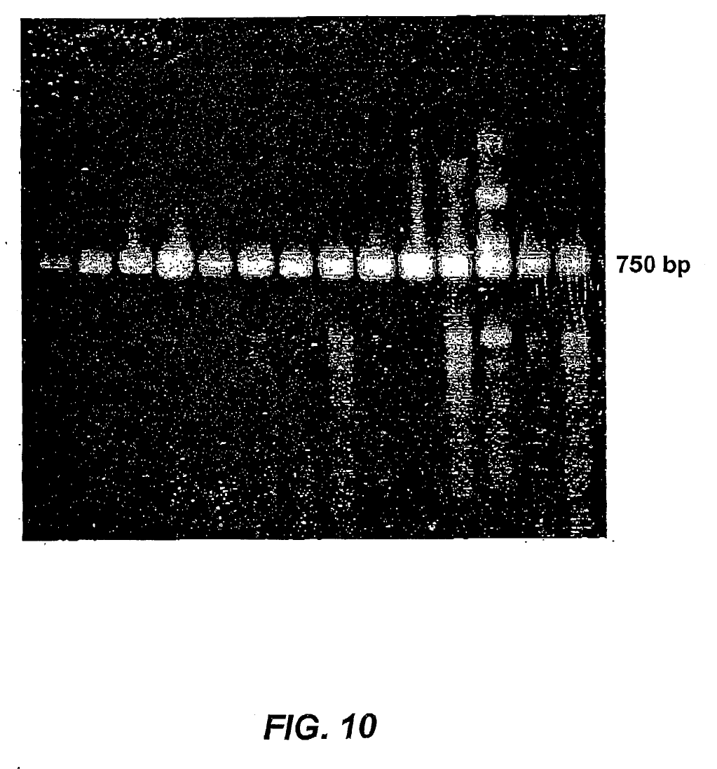

Experiments to show the presence of the introduced green fluorescent protein gene (sgfpS65T) in the transgenic plants were conducted using the polymerase chain reaction (PCR). The data are presented in FIG. 10. A rapid DNA release technique was used to extract DNA from a number of individual transgenic plant lines or alternatively a plant genomic DNA extraction technique (described in Example 11). For the rapid DNA release technique, small amounts of young leaf tissue (approximately 15 mm²) were harvested and ground in liquid nitrogen before resuspending in template preparation solution (TPS; 100 mM Tris-HCl pH9.5, 1 M KCl, 10 mM NaEDTA). This was incubated at 95° C. for 10 minutes before quenching on ice and chloroform/isoamyl extraction and centrifugation at 13,000 rpm for 5 minutes. A 1.5 dilution of aqueous phase was used for PCR.

PCR was conducted using an PCR reagents (Ambion Inc.) and primers (5′-3′) ATG GTG AGC AAG GGC GAG GAG (SEQ ID NO: 1) and (5′-3′) TTA CTT GTA CAG CTC GTC CAT (SEQ ID NO:2) which amplify an approximate 750-bp of sgfpS65T coding region. No band was amplified in PCR control samples or using DNA from sugarcane cv. Q165 untransformed plants. A 750-bp band corresponding to the sgfpS65T coding region was amplified in over 50 lines of GFP-positive transgenic plants, examples of which are shown in FIG. 10.

### EXAMPLE 10

### Confirmation of the Presence of Introduced sgfpS65T Integrated in Genomic DNA of Transgenic Plants

Plant genomic DNA was extracted using a proteinase K DNA extraction technique. To extract genomic DNA, 2 g of young leaf tissue was harvested from individual lines of transgenic plants and frozen in liquid nitrogen before being ground to a fine powder. This was immersed in 0.1 mg/mL Proteinase K solution in buffer S (100 mM Tris-HCl pH8.5, 100 mM NaCl, 50 mM EDTA, 2% SDS and 10 mM DTT) and incubated at 55° C. for 1 hr. This was followed by phenolchloroform extraction then by ethanol precipitation. Crude DNA was resuspended in TE buffer followed by RNasc treatment to degrade any RNA present in the samples. DNA samples were then further phenol:chloroform extracted and ethanol precipitated once more before the DNA was resuspended in TE buffer for subsequent testing.

Plant genomic DNAs were digested with the restriction enzyme HindIII or BamHI. There was only onie HindIII site in the plasmid pGEM.Ubi-sgfpS65T which was used in the transformation procedure via microprojectile bombardment. This site was also positioned at the 5′ start site of e Ubi promoter region of the Ubi-sgfpS65T-nos gene. There is also a BamHI site 5′ of the sgfpS65T coding region in plasmid pGEM.Ubi-stS65T. After digestion, the DNAs were electrophoresed through a 0.9% THE gel and transferred. to a nylon membrane, (Hybond N+) by standard techniques introduced by Southern, 1975, J. Mol. Biol. 98 503-527. A region of the sgfpS65T coding region from the plasmid pGEM.Ubi-sgS65T-nos used in transformation was amplified by PCR for use as a probe This was achieved using an PCR reagents (Ambion Inc.) and primers (5′-3′) ATG GTG AGC AAG GGC GAG GAG (SEQ ID NO:3) and (5′-3′) TTA CTT GTA CAG CTC GTC CAT (SEQ D) NO:4) to amplify an approximate 750 bp of sgS65T coding region. This was gel pmified and isolated using a Qiagen Minielute gel extraction kit according to the manufacturers instructions. This DNA fragment (approximate 750 bp s6S65T) was denatured and labelled with ³²P[α-dCTP] using a Rediprime DNA labelling kit (Amershan) and used as a labelled probe. Hybridisation was performed according to Sambrook et al.,1989, Molecular Cloning: a laboratory manual, 2^{nd} ed. Cold Harbour Laboratory Press, Cold Spring Harbour, N, in 5×SSPE, 5×Denhardt's solution, 0.1% SDS, 0.1 mg mL⁻¹denatured fish sperm, 0.1 g mL⁻¹ dextran sulphate for 20 h before membranes were washed with 0×SSPE, 0.1% SDS solution at 650° C. Hybridizing bands were detected after 1-4 days exposure to a Molecular Dynamics Storage Phosphor screen and imaged on a Molecular Dynamics Storm840 phosphoimager using Strom scanner control version 5.0 software

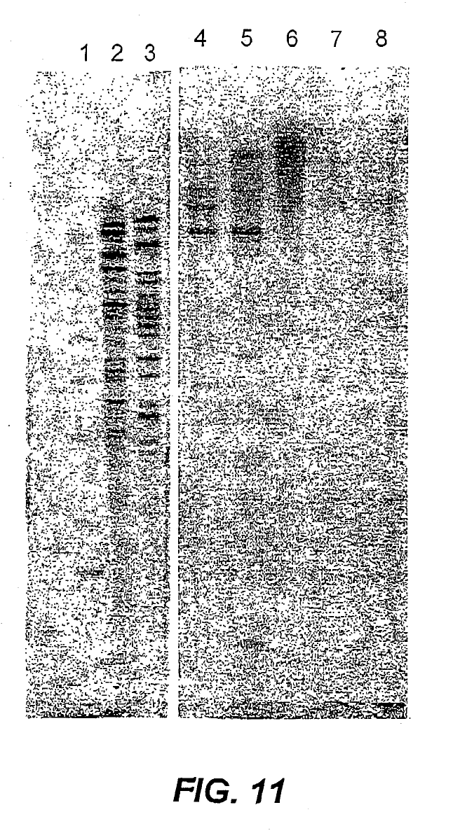

FIG. 11 shows the presence of s,gfpS65T sequences integrated into the genomic DNA of 3 transgenic sugarcane lines: #10, #13 and #F10. Multiple insertions have occurred shown by the different hybridising band sizes. The integration events are also different in the three transgenic lines; as a result of the random integration patterns which occurs with microprojectile bombardment. Hybridizing band patterns for transgenic line #F10 in lanes 4 and 5 result from digestion with HinDIII and BamHI respectively. Lanes 7 and 8 show no hybridizing bands in untransformed cv. Q165.

### EXAMPLE 11

### Agrobacterium-Mediated Transformation of Sugarcane

Agrobacterium strains LBA4404, AGL0, AGL1 and EHA101 were transformed with the binary vector plasmid pBIN.Ubi-sgfpS65T (Elliott et al., 1998, supra). Agrobacterium stocks were then grown on LB solid medium containing 50 mg L⁻¹ kanamycin sulphate to select for the presence of the binary vector plasmid. Inoculating cultures were ten grown on LB, MG/L or YEP pH 5.4 solid medium or in liquid medium without selection at 28° C. for 2-3 nights. Various vir gene-inducing agents were added to medium to stimulate vir gene expression and promote Agrobacterium virulence, including 100 μM acetosyringone or 100 μL petunia extract or 10 mM glucose, Thin sections of sugarcane cv. Q165 stems were excised as described for microprojectile bombardment. These were placed “top dower” on MS medium containing 4 μM BA and 10 μM NAA and pre-cultured for 3 or more days prior to inoculation. Before inoculation, the sugarcane thin sections were dried in a laminar flow to induce some plasmolysis and then transferred to a 50 mL sterile tube. Agrobacterium cells were washed from plates or diluted from liquid cultures using LB pH 5.4 containing 100 liM acetosyringone. Agrobacterium cultures were diluted to an OD₆₀₀ so of approximately 0.2-0.8. Diluted Agrobacterium cultures were added to the tubes containing the explants until all explants were immersed and gently shaken for 20 minutes. Vacuum infiltration which is a standard procedure was sometimes applied.

The infected tis sections were then blotted dry and cultured in the same orientation as prior to inoculation for 2 to 4 days on shoot regeneration medium containing MS, 4 M BA, IO aM NAA and l00 F acetosyringone at 24° C. After this period, the infected explants were washed in sterile water and plated again in the same orientation as before onto MS medium supplemented with 4 liM BA, 10 μM NAA, 150 mg L⁻¹ Timentin (Smith Kline Beecham) for 4-10 days. Explants were then plated onto MS medium supplemented with 4 μM BA, 10 μM NAA, 150 mg L⁻¹ Timentin and 150 mg L⁻¹ paromomycin sulfate.

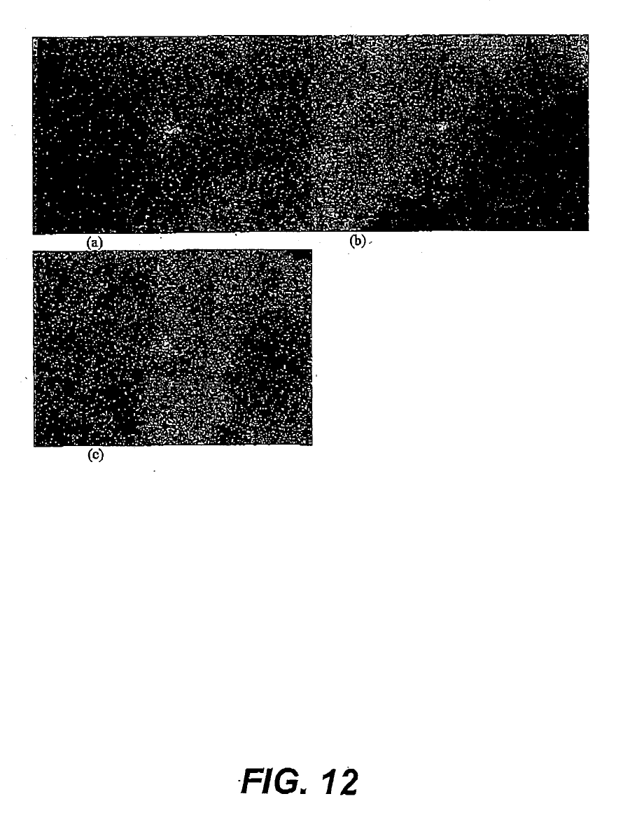

In an alternative procedure, thin section explants of cv. Q165 were initiated in the “top down” orientation on MS medium containing b 4 μM BA, 10 μM NAA and 10 μM chloropbenoxyacetic acid (CPA) for 3 or more days prior to inoculation. The inoculation procedure was then carried out as described above, except that 10 4 μM CPA was included in the culture medium in addition to the 4 μM BA and 10 μM NAA. Regenerating cells were monitored for the presence of GFP-positive cells. Examples of GFP-positive transgenic cells after Agrobacterium cocultivation are provided in FIG. 12. Stable integration of Ubi-sgfpS6ST -nos was noted from the division of transgenic GFP+cells on the surface of the thin sections and by the persistence of expression for greater than 12 weeks after the cessation of cocultivation.

### EXAMPLE 12

### Wheat Transformation

Stem thin section explants were excised from approximately 20 cm tall wheat cv. Janz plants in the regions of nodes below the immature floral tissue and in the region of the immature floral tissue. Thin sections (2-4 mm) were placed “top-down” on FM media containing 01 g/L L-ascorbic acid, 0.15 g/L citric acid and 10 μM or 20 μM CPA. These were cultured for 3 to 5 days before bombardment at 2000 -8000 kPa ]Bombardment conditions are as described for sugarcane transformation whereby a 130 μl precipitation mixture was utilised, plasmids pGEM,Ubi-sXS6ST and pUKN were co-precipitated and pressure was as stated above. Transient transformation frequency was recorded at 3 days after bombardment.

Explants were transferred to EM media with 10 μM or 20 μM CPA and 100 mg L⁻¹ paromomycin sulfate 12-14 days after initiation at approximately 9 -11 days after bombardment. After another 8 days explants were transferred Lo EM media containing 5 μM zeatin and 100 mg L⁻¹ paromomycin sulfate.

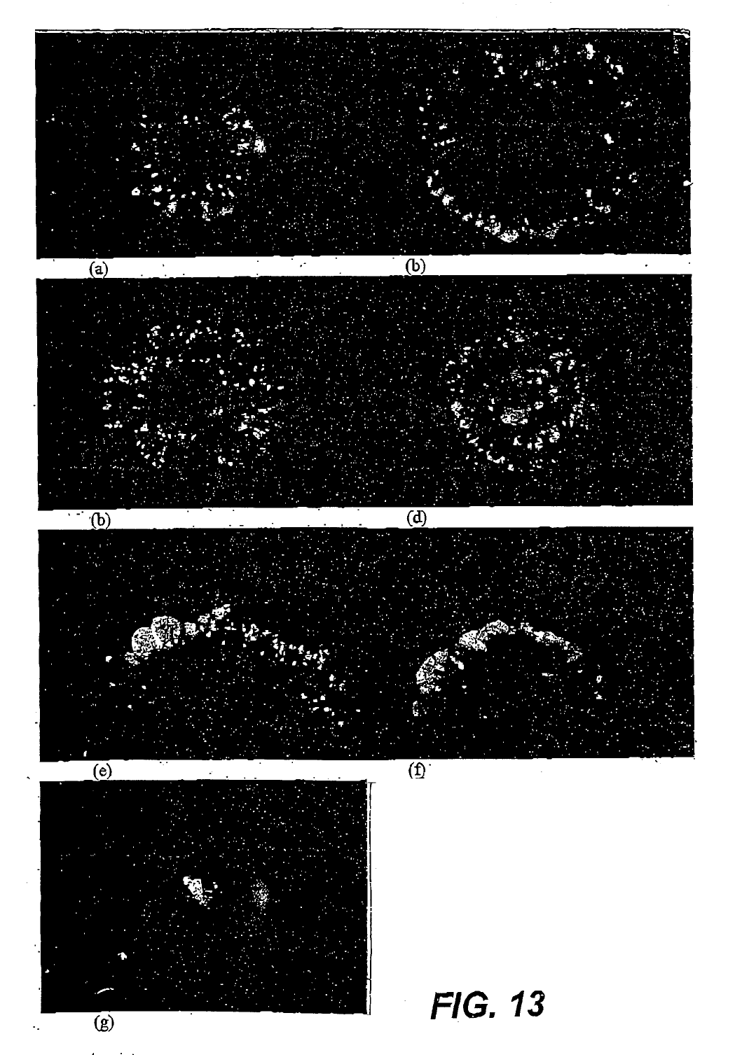

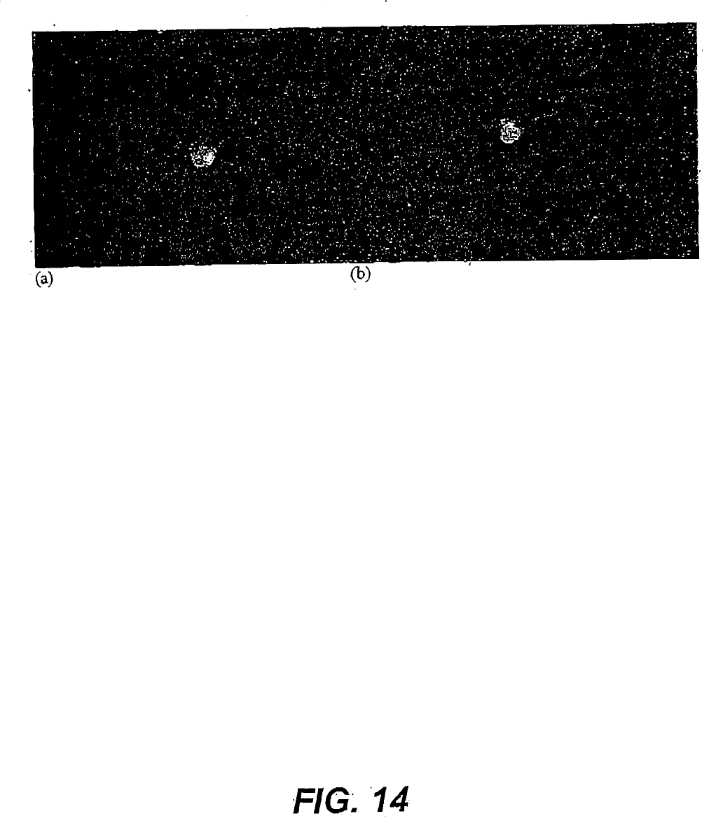

Culturing continued on this media combination with regular sub-culturing. During this tie, clumps of transgenic G⁰T-positive cells formed on the thin sections, as shown in the FIGS. 13 and 14 A number of small GFP-positive wheat apices or embryo-like structures were formed on the surface of the wheat TS explants, Plants can be regenerated from these structures on EM supplemented with 5 μM zeatin and 100 mgL⁻¹ paromomycin sulfate such as according to thc methods described in Wernicke & Milkovits 1984,. J. Plant Physiol. 115 49-58 and Wernicke & Milkovits, 1986, Protoplasma 131 131-141.

### EXAMPLE 14

### Sorghum Transformation

Thin section sorghum explants (1-4 mm) were obtained from developing leaf sheaths (the innermost 3-4 whorls at the shoot tip). These explants were placed “top down” in orientation on MS medium supplemented with 10 μM or 20 μM chlorophenoxyacetic acid (CPA) alone or in the presence of 4 μM benzylaminopurine (BA) and cultured at 28° C. After 46 days, the explants were transferred to the same media but wit 0.2M mannitol and 0.2M sorbitol in preparation for bombardment. The explants were bombarded with pGEM.Ubi-sgS65T and pUKN using conditions described previously for sugarcane. Transient expression of Ubi-sgfpS65T-nos was recorded in cells on the surface of the sorghum thin section explants.

It will be understood that the invention is not limited to that which is described in detail herein, and that a varied of other embodiments may be contemplated which nevertheless fall within the scope and spirit of the invention.

All computer programs, scientific literature and patent literature referred to in this specification are incorporated herein by reference.

1. A method of producing a transgenic monocotyledonous plant including the steps of:
(i) culturing a thin section explant from a monocotyledonous plant in the presence of an auxin and, optionally, a cytokinin;
(ii) transforming said thin section explant with an exogenous nucleic acid and, optionally, with a selection marker nucleic acid; and
(iii) selectively propagating a mature transgenic plant from the transformed explant obtained in step (ii).

2. The method of claim 1, wherein step (iii) includes the sequential .steps of:
a) culturing the transformed explant in medium comprising a selection agent together with an auxin and cytokinin; and
(b) culturing the transformed explant in medium comprising a selection agent in the absence of an auxin and a cytokinin.

3. The method of claim 1, wherein the thin section is oriented during culture at step (i) so that a basal surface is substantially not in contact with a culture medium.

4. The method of claim 1, wherein the thin section explant is 1.0-1 0.0 mm thick.

5. The method of claim 4, wherein te thin section explant is 1.0-6.0 mm or 2.0-3.0 nm thick.

6. The method of claim 1, wherein the thin section is of leaf whorl.

7. The method of claim 1, wherein the thin section is of inflorescence.

8. The method of claim 6 or claim 7; wherein the thin section is obtained from sugarcane,

9. The method of claim 1, wherein the tin section is of immature floral material.

10. The method of claim 9, wherein the thin section is obtained from wheat,

11. The method of claim 1, wherein the monocotyledonous plant is a member of the Graminae family.

12. Thc method of claim 11, wherein the monocotyledonous plant is sugarcane, wheat or sorghum

13. The method of claim 1, wherein the duration of culture at step (i) prior to transformation is 1-6 days.

14. The method of claim 1, wherein at step (ii), the transformed explant is cultured prior to selective propagation at step (iii) for a period of 4-15 days in the absence of a selection agent.

15. The method of claim 14, wherein the period is 5-10 days.

16. The method of claim 14 or claim 15, wherein a cytokinin and an auxin are present during the respective periods of 4-15 days or 5-10 days.

17. The method of claim 1 wherein the auxin is selected from the group consisting of x-napthaleneacetic acid BAA) or p-chlorophenoxyacetic acid (CPA).

18. The method of claim 1, wherein a cytokinin is present.

19. The method of claim 18, wherein the cytokinin is selected from the group consisting of kinetin (KIN), zeatin and N⁶-benzyladenine (BA),

20. The method of claim I wherein transformation at step (ii) is by microprojectile bombardment.

21. The method of claim I wherein transformation at step (ii) is Agrobacterium-mediated.

22. A method of producing a transgenic sugarcane plant including the steps of:
(i) culturing a thin section explant from a sugarcane plant in the presence of N⁶-benzyladenine (BA) and α-napthaleneacetic acid BAA) for 24 days, wherein the thin section explant is oriented during culture at step (i) so that a basal surface is substantially not in contact with the culture medium;
(ii) transforming said thin section explant by microprojectile bombardment with an exogenous nucleic acid and a selection marker nucleic acid;
(iii) culturing the microprojectile bombarded explant for 4-15 days in the presence of BA and NAA and in the absence of a selection agent; and
(iv) selectively propagating a mature transgenic sugarcane plant from the transformed explant obtained in step (iii).

23. A method of producing a transgenic wheat plant including the steps of:
(i) culturing a thin section explant from a wheat plant in the presence of p-chlorophenoxyacetic acid (CPA) for 3-5 days, wherein the tin section is oriented during culture at step (i) so that a basal surface is substantially not in. contact with the culture medium
(ii) transforming said thin section explant by microprojectile bombardment with an exogenous nucleic acid and a selection marker nucleic acid;
(iii) culturing the microprojectile bombarded explant for 9-11 days in the presence p-chlorophenoxyacetic acid (CPA) and in the absence of a selection agent; and
(iv) selectively propagating a mature transgenic wheat plant from the transformed explant obtained in step (iii).

24. A method of producing a transgenic sorghum plant including the steps of:-
(i) culturing a thin section explant from a sorghum plant in the presence of p-chlorophenoxyacetic acid (CPA) and, optionally, N⁶-benzyladenine (BA) for 4-6 days, wherein the thin section is oriented during culture at step (i) so that a basal surface is substantially not in contact with the culture medium;
(ii) transforming said thin section explant by microprojectile bombardment with an exogenous nucleic acid and a selection marker nucleic acid;
(iii) culturing the microprojectile bombarded explant in the presence p-chlorophenoxyacetic acid (CPA) and in the absence of a selection agent; and
(iv) selectively propagating a mature transgenic wheat plant from the transformed explant obtained in step (iii).

25. A transformed monocotyledonous plant cell or tissue produced at step (ii) of claim 1.

26. A transgenic monocotyledonous plant produced according to claim 1.

27. Reproductive material obtained from the transgenic plant of claim 26.

28. A transgenic Graminae plant according to claim 26.

29. A transgenic sugarcane plant according to claim 28

30. A transgenic cereal plant according to claim 28.

31. A transgenic wheat plant according to claim 30.

32. A transgenic sorghum plant according to claim 30.


SEQUENCE LISTING (4 sequences)

SEQ ID NO: 1 - DNA, 21 bp, Artificial Sequence
  PCR Primer
atggtgagcaagggcgaggag

SEQ ID NO: 2 - DNA, 21 bp, Artificial Sequence
  PCR Primer
ttacttgtacagctcgtccat

SEQ ID NO: 3 - DNA, 21 bp, Artificial Sequence
  PCR Primer
atggtgagcaagggcgaggag

SEQ ID NO: 4 - DNA, 21 bp, Artificial Sequence
  PCR Primer
ttacttgtacagctcgtccat